(19) United States
(12) Patent Application Publication
(10) Pub. No.: US 2006/0191319 A1
(43) Pub. Date: Aug. 31, 2006

(54) Electronic nose for chemical sensing

(76) Inventor: Pradeep U. Kurup, Nashua, NH (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 11/305,303

(22) Filed: Dec. 16, 2005

Related U.S. Application Data
(60) Provisional application No. 60/637,000, filed on Dec. 17, 2004.

(51) Int. Cl. G01N 33/497

(52) U.S. Cl. 73/23.34

(57) ABSTRACT

An apparatus and method for detecting selected chemical compounds or elements is presented. A sensor array is exposed to one or more odors, for example. The outputs of the sensor constitute input to a pattern recognition system. Embodiments of the invention can also be trained for detecting odors in a soil column, for detecting odors associated with different varieties of food such as coffee beans, and for detecting odors associated with substantially any odorant.

### CROSS REFERENCE TO RELATED APPLICATIONS

The present application claims priority to U.S. Application No. 60/637,000 filed on Dec. 17, 2004, the entire contents being incorporated herein by reference.

### STATEMENT REGARDING FEDERALLY SPONSORED RESEARCH OR DEVELOPMENT

This invention was supported, in whole or in part, under grant number 9875037 from the National Science Foundation. The Government has certain rights in the invention.

### BACKGROUND OF THE INVENTION

Odorants are substances capable of producing odors. Examples of odorants are, but are not limited to, chemicals such as benzene, toluene, and ethylbenzene, foods such as coffee, meat, and produce, and other substances such as natural gas, perfume, and smoke. Odors associated with an odorant may be indicative of its basic existence, as in the case of a chemical, or odors may indicate that the odorant has undergone a change such as would occur with food spoilage.

In many applications it is desirable to detect and identify odors or the substances that cause them. For example, a food processing plant may want to detect spoiled food before it leaves the plant or an environmental remediation contractor may want to identify odorous compounds contained in a soil sample at a contamination site. These chemicals can sometimes be identified by humans or specially trained animals and/or using machines. An example of machine-based system that can be used for odor classification is gas chromatography. When detecting potentially hazardous odors, machine-based classification may be the only desirable option. These machine-based classification systems are often time consuming to use and their size and complexity make them undesirable for field use.

There exists a need for an automated air or fluid borne chemical detection system that is portable and that is further capable of performing near real-time classification of odors. In addition, it is desirable that a system for detecting such chemicals be capable of classifying odors associated with certain chemicals and food products.

### SUMMARY OF THE INVENTION

The system and method of the present invention relates to the use of a sensor system in combination with a processing system for identifying the presence of certain substances. The substances of interest include certain airborne chemicals that cause odors. A preferred embodiment of the invention uses a pattern recognition system such as a neural network to analyze data and identify chemicals contained in the fluid or gas exposed to the sensor system. Such a system can include a pattern learning module by which reference data can be learned and stored electronically and compared with sensor output signals to identify and quantitatively measure chemicals being detected. The system can be used as a portable chemical analysis system to identify chemicals present at a location and provide a quantitative measurement in real time.

A preferred embodiment of the invention is used for a probe such as a penetrometer to detect the presence of chemicals in subsurface or underground locations. The probe can have a diameter of 2 inches or less and can be launched from a push or drill rig, or truck with push rods used to advance the probe into soil or sediment. The system can be used for chemical identification and monitoring in environmental contamination. Various systems and methods of detection can be used in the collection of samples and the measurement of the chemical constituents in those samples. For example, the chemical sensor can be combined with temperature and humidity sensors in the probe or instrument housing to characterize the collection site. Subsurface samples can be collected using a purge and trap system, a vacuum extraction system or by diffusion through a heated semi-permeable membrane. Fluid sample testing can be used that can include an underwater environmental monitoring sampling or detector system. Optical, metal oxide sensors, conducting polymer sensors, chemoresistive, surface acoustic wave or quartz microbalance sensors or a hybrid array using a selected combination of these sensors can also be used to detect chemicals present in the sample.

The pattern learning and recognition system can include various systems and methods including artificial neural networks such as multi-layer perception (MLP), generalized regression neural network (GRNN), fuzzy inference systems (FIS), self-organizing map (SOM), radial bias function (RBF), genetic algorithms (GAS), neuro-fuzzy systems (NFS), adaptive resonance theory (ART) and statistical methods such as principal component analysis (PCA), partial least squares (PLS), multiple linear regression (MLR), principal component regression (PCR), discriminant function analysis (DFA including linear discriminant analysis (LDA), and cluster analysis including nearest neighbor.

In another preferred embodiment, the invention can be used to identify or characterize foods having a certain olfactory pattern such as coffee. The system can be used during coffee production or other food processing operations, for example, to monitor processing conditions, to avoid product deterioration, contamination or damage during processing, storage or transport.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 illustrates an exemplary method for detecting an odor using an artificial neural network based processor;

FIG. 2A illustrates a schematic diagram of an electronic cone tip being used to monitor odors in a soil column;

FIGS. 2B and 2C illustrate exemplary penetrometer configurations useful for measuring odors contained in a soil column;

FIG. 2D is an illustration is an illustration of an underwater sample collection system in accordance with a preferred embodiment of the invention.

FIGS. 3A and 3B illustrate exemplary topologies for an artificial neural network capable of processing odors obtained using a sensor array;

FIG. 3C illustrates an exemplary implementation of a hidden neuron used in conjunction with the network topologies of FIGS. 3A and 3B.

FIG. 4 illustrates exemplary signatures for compounds detected using a sensor array and artificial neural network in accordance with an embodiment of the invention;

FIG. 5 illustrates an exemplary implementation of a prediction mode that can be used in conjunction with embodiments of the invention;

FIG. 7 illustrates an exemplary data plot showing results of a principal component analysis in accordance with an aspect of the invention;

FIG. 8 illustrates a schematic representation of an embodiment useful for detecting and classifying odors associated with coffees;

FIG. 9 illustrates a schematic representation of an embodiment used for measuring odors associated with a plurality of coffee beans;

FIG. 10 illustrates a plot showing exemplary results obtained by measuring samples of coffee beans with an embodiment of the invention;

FIG. 11 illustrates a schematic representation of a computer architecture that can be used for implementing the functionality of an artificial neural network used in embodiments of the invention; and

FIG. 12 illustrates an exemplary method for detecting and classifying odors using embodiments of the invention.

### DETAILED DESCRIPTION OF THE INVENTION

Embodiments described herein detect and classify certain chemicals in a fluid medium using a neural network based processor. The substances or chemicals of interest are detected by the system using electronic and/or electromechanical sensors. The sensors convert the detection of certain substances into electrical signals which are conveyed to a pattern recognition system, such as neural network, and a result is generated.

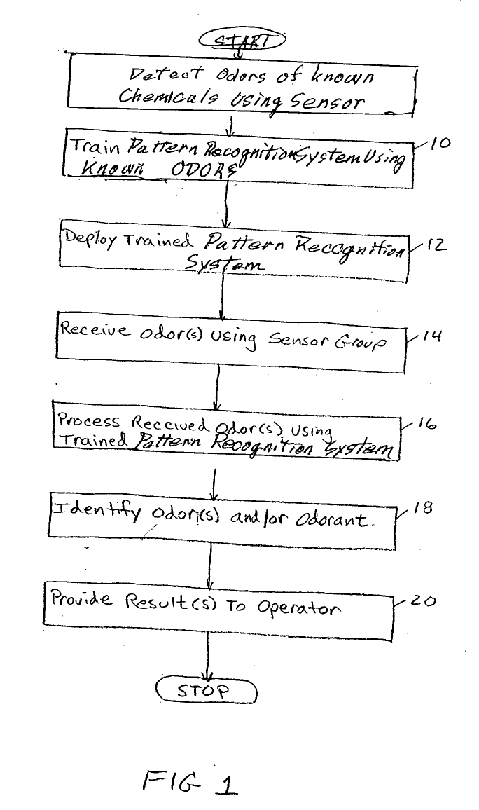

FIG. 1 illustrates an exemplary method for classifying an odor using a preferred embodiment. The method starts with training of a neural network, for example, using known odors (per step 10). Once the neural network is trained, it is deployed (per step 12). The deployed system receives one or more odors using a sensor group (per step 14). The received odors are processed using the neural network which, in a preferred embodiment, is an artificial neural network (per step 16) and one or more results are generated. The results provide identification of odorants based on received odors, or vapors (per step 18). These results are provided to an operator in substantially real-time (per step 20).

As used herein real-time refers to an event or a sequence of steps, such as are executed by a processor that are perceivable by a user or observer at substantially the same time that the event is occurring or that the steps are being performed. By way of example, if the neural network of FIG. 1 receives an odor, the system produces a result at substantially the same time that the odor was sensed. This real-time processing can have some time delay associated with converting sensed odors to electrical signals for input to the neural network and further associated with the processing of data by the neural network; however, any such delay is less than 1 minute and typically no more than a few seconds.

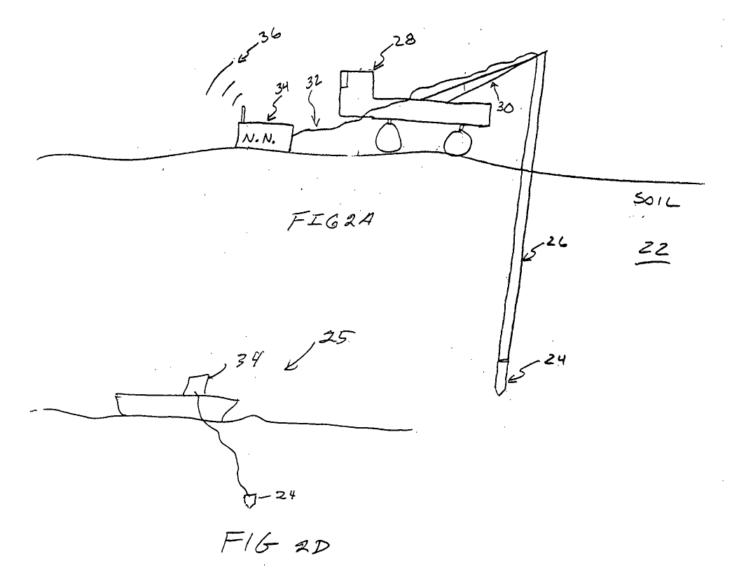

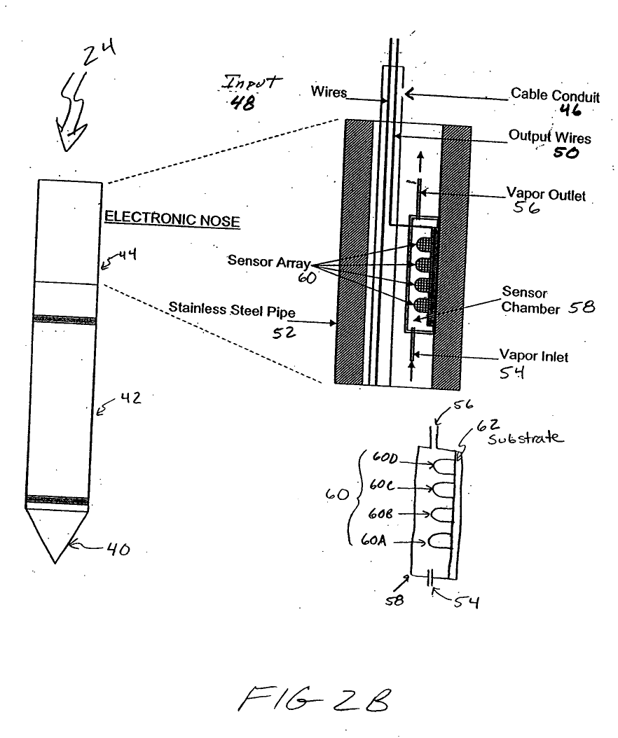

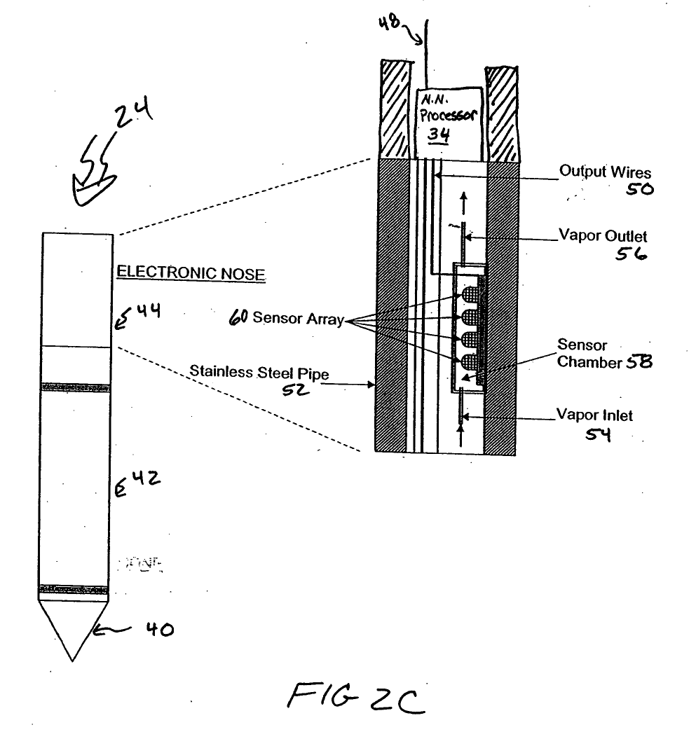

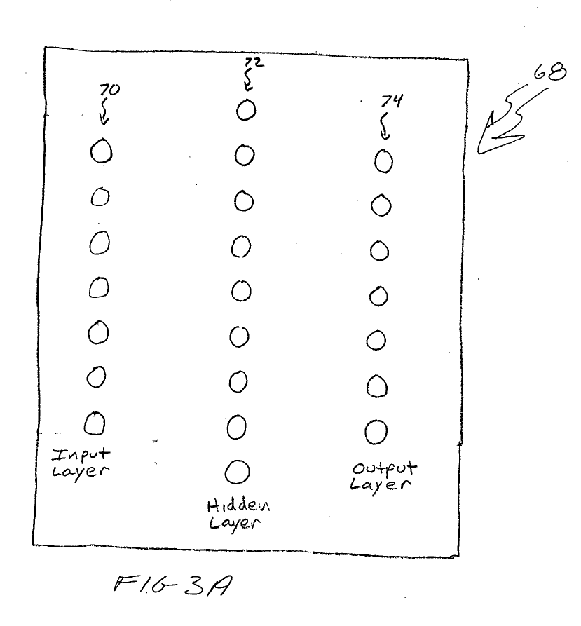

A preferred embodiment of an electronic odor sensing apparatus is useful for detecting volatile organic compounds (VOCs) in soil. For example, this embodiment can be used for real-time site assessment and monitoring activities associated with hydrocarbon contamination in soil. FIG. 2A illustrates an embodiment of a field measurement system capable of detecting and classifying odors associated with soil borne contaminants. A direct push probe 24 is connected to a sensing instrument module 34 and is driven into a soil column 22 using a hollow push rod 26 and a vehicle mounted hydraulic ram 30. A vehicle 28, such as a truck, includes a support carriage for retaining and operating ram 30. Probe 24 contains sensors and electronics for detecting odors and for monitoring other useful parameters. Probe 24 is further described in conjunction with FIGS. 2B and 2C.

Probe 24 may be coupled to a pattern recognition system such as a neural network processor 34 by way of a data cable 32 running through an inner channel of rod 26. Rod 26 may include, for example, stainless steel piping. Neural network processor 34 may store input data and processed results therein or processor 34 may convey the results to a remote location using, for example, a free-space wireless radio frequency (RF) transmitter 36.

FIG. 2B illustrates a preferred embodiment of Probe 24 in more detail. Probe 24 is made up of a cone tip which is coupled to transition piece 42. Cone 40 is conical in shape and operates to facilitate penetration of probe 24 into soil column 22. Cone 40 is typically made from a hard and durable material such as treated steel, titanium, or the like. In alternative embodiments, cone 40 can be replaced with a drill bit for facilitating penetration through rock or other hard substances in soil column 22. If a drill head is used, rod 26 can be configured such that only the drill head rotates or so that rod 26 also rotates.

Transition section 42 couples cone 40 to sensor housing 44. Transition section 42 is cylindrical in shape and may be substantially solid or it can be hollow. In a preferred embodiment transition section 42 is fabricated from stainless steel. Transition section 42 is coupled to sensor housing 44 at its upper end.

Electronics housing 44 can include a pipe having an outer diameter matching that of transition section 42. Electronics housing has an internal volume that houses a gas or vapor inlet 54, a sensor chamber 58 housing a sensor array 60, and a vapor outlet 56. In addition, a cable conduit 46 containing input wires 48 and output wires 50 may be provided. Vapor inlet 54 consists of a tube which allows a gas or vapor, containing odors, to enter sensor chamber 58. Once the vapor is inside sensor chamber 58, it is exposed to sensor array 60. Sensor array 60 includes a plurality of sensors 60A-60D that are each capable of detecting one or more odor types. In a preferred embodiment, sensors 60A-60D are selected so that together they provide the ability to detect a desired range of odorant types. Sensor array 60 may be replaceable so that probe 24 can be quickly adapted for detecting different classes and types of odorants.

Sensors 60A-60D may consist of, for example, optical sensors, metal oxide sensor (MOS) elements, surface acoustic wave (SW) elements, electrically conducting inorganic polymer elements, electrically conducting organic polymer elements, and quartz crystal micro-balance elements. In addition, sensors 60A-60D may consist of other element types capable of having a varying electrical behavior, such as optical conductivity, frequency shift, etc., upon exposure to a gas or vapor. Sensors 60A-60D may employ techniques such as pre-heating a vapor before sensing in order to enhance a sensor's response characteristics to an expected vapor type. Sensor chamber 58 can also include a gas or vapor outlet 56 for facilitating egress of vapors after passing across sensor array 60.

Input wires or cables 48 convey power, bias signals, programming/gain control commands, and the like, to sensor array 60. Output wires convey sensor output signals, error data, and auxiliary data to equipment located above soil column 22.

Auxiliary data may include, among other things, temperature data from a temperature probe, vapor pressure data, humidity data, depth data, vibration data, soil conductivity data, soil resistance data, acoustic data, pore water pressure data, and soil moisture data.

FIG. 2C illustrates a preferred embodiment wherein probe 24 further includes a neural network based processor 34. In some applications, it may be desirable to have sensor data processed proximate to sensor array 60. Proximate processing may be desirable when probe 24 is attached to a rod 26 that is not equipped to pass signals to processing devices located proximate to vehicle 28. In the embodiment of FIG. 2C, transition section 42 can contain a battery for powering sensor array 60 and processor 34.

Probe 24 may include other types of sensors in addition to odor, or vapor, sensor array 60. For example, conventional sensors useful in making soil-based measurements may be used. Alternative embodiments may include a load cell for determining a force applied to probe 24, pressure transducers for measuring pore-water pressures, geophones and/or accelerometers for recording arrival times of compression and shear waves generated at the surface, conductivity sensors for measuring the electrical conductivity of the soil, electrical resistivity/domain reflectometry for measuring relationships between a soil dielectric constant and moisture constant, vision sensors for visually observing a soil column, and the like.

In addition, probe 24 may employ techniques such as purge and trap, vacuum extraction using a pump or piston assembly, or diffusion through a heated semi-permeable membrane to facilitate extraction of vapor samples from soil column 22.

Illustrated in FIG. 2D is another preferred embodiment of the invention in which a water borne platform or vessel 25 is used to support an instrument module 34 that is connected via electrical cable to an immersed probe 24 that detects selected compounds in water. This system can be used for environmental testing or monitoring, for example.

In a preferred embodiment, sensor data is provided to an artificial neural network (ANN). An ANN is a data processing architecture making use of highly interconnected nodes, referred to as neurons, for mapping a complex input pattern with a complex output pattern. ANN's have the capacity to learn, or be trained, from example input-output training data sets. The potentially numerous interconnections among the neurons in conjunction with the use of adaptive weighting functions coupling the neurons can yield tremendous computational power. ANN's tend to be tolerant of noisy and fuzzy data thus making ANN's more robust than many types of mathematical models. Embodiments disclosed herein make use of a feedforward neural network; however, other neural network architectures such as fuzzy ARTMAP can be used without departing from the spirit of the invention. In particular, a feedforward multi-layered perceptions trained by back-propagation algorithms based neural network architecture may be used.

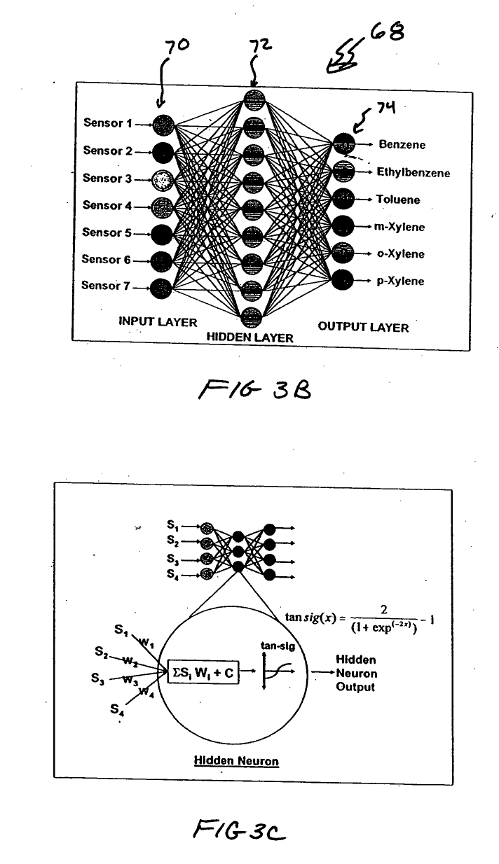

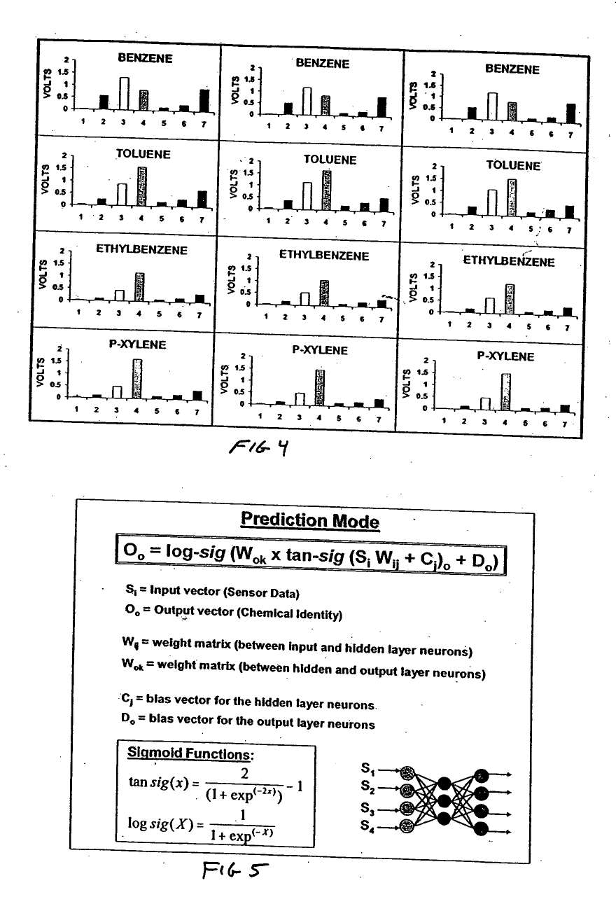

FIG. 3A illustrates an exemplary ANN architecture 68 that can be employed for processing data from sensor array 60. In general, the architecture 68 utilizes 3 layers of neurons. The first layer is an input layer 70 containing a plurality of input neurons for receiving input data such as odors from a soil sample. Hidden layer 72 contains a plurality of neurons wherein each hidden neuron is coupled to every input neuron. Output layer 74 includes a plurality of output neurons organized such that each output neuron is coupled to every hidden neuron.

FIG. 3B illustrates input layer 70, hidden layer 72 and output layer 74 along with exemplary interconnections. In addition, the architecture 68 of FIG. 3B illustrates input sensors for detecting vapors along with a range of exemplary output types. The architecture 68 contains seven input sensors, or input neurons, each of which is mapped to every hidden neuron. In the feedforward implementation, the hidden layer 72 performs processing on data received from input layer 70 before making outputs available to output layer 74. The architecture 68 of FIGS. 3A and 3B illustrate a single hidden layer; however essentially any number of hidden layers can be used with the number of neurons in each layer being limited only by processing power and available system memory.

The manner in which the various hidden neurons 72 process input data dictates how the input data is transformed. In a preferred embodiment of architecture 68, a modifiable weight is associated with each neuron interconnection. The modifiable weight is analogous to a synapse connecting neurons in a human brain. The hidden neurons perform non-linear transformations on the input data. In particular, each hidden neuron transforms the sum of the weighted inputs it receives along with a bias using a transfer function which is referred to as an activation function.

FIG. 3C illustrates an exemplary transfer function as used with preferred embodiments described herein. Hidden neurons may typically use linear, long-sigmoid or log-sigmoid functions. In addition, every hidden neuron and every output neuron may have its own modifiable bias term in order to facilitate the universal approximation capability of the multilayer perceptrons.

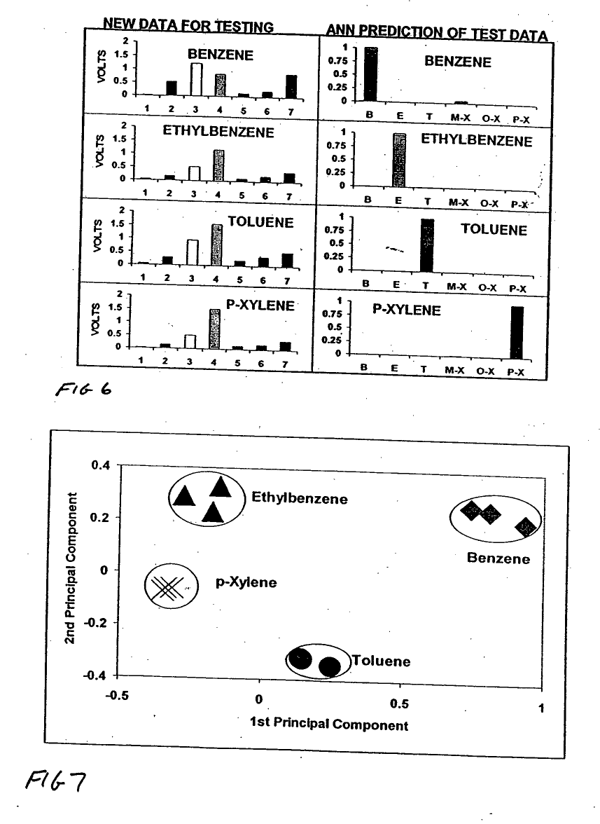

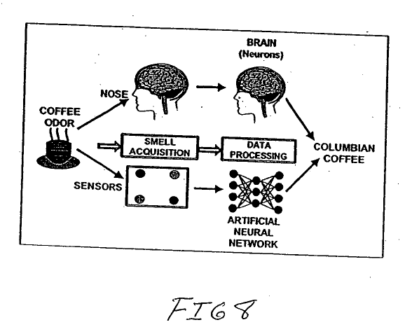

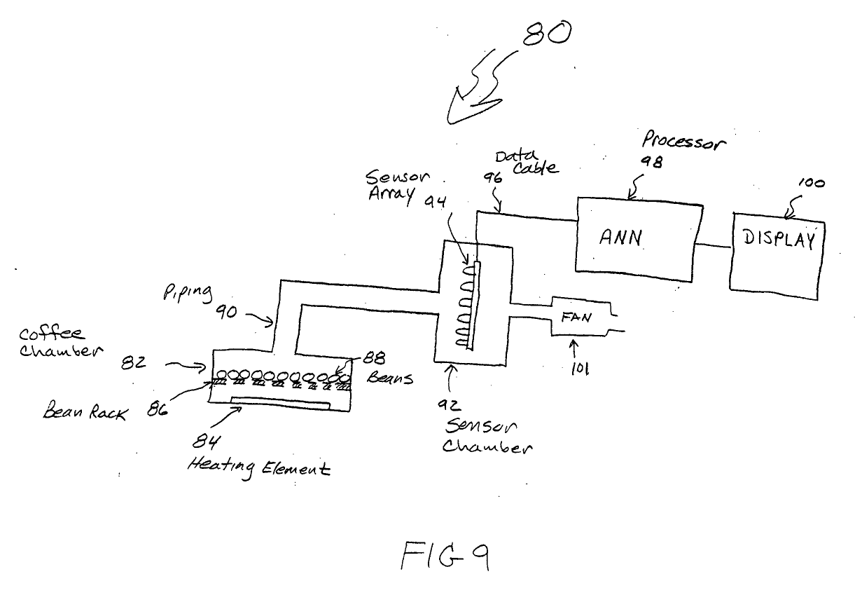

The inputs to the input layer of neurons are the sensor outputs corresponding to chemical fingerprints (FIG. 4). If there are seven sensors in the array, the input layer will have seven neurons. For the architecture shown, the number of neurons in the output layer corresponds to the number of chemicals that the electronic nose is trained to identify. Alternatively the output layer may have a fixed number of neurons, and the chemicals may be identified by a number assigned thereto, respectively. The number of neurons in the hidden layer is determined by training several networks with different numbers of hidden neurons and comparing the predicted results with a desired output. Embodiments discussed herein employed anywhere from four to ten neurons; however, methods disclosed herein can employ larger numbers of neurons if desired. Using too few hidden neurons may result in large training errors, as well as errors during testing, due to underfitting and high statistical bias. On the other hand, using too many hidden neurons might give low training errors while still producing high testing errors due to overfitting and high variance. Excessive training so as to obtain very low errors may also result in overtraining (or overfitting), where a network's performance becomes worse instead of better after a certain point during training. Overtraining causes the network to memorize the example training patterns (including all of their peculiarities) to such an extent that it is unable to generalize for new data. Therefore, preferred embodiments utilize training that does not result in overfitting.

ANNs like people, learn by examples. Training of a neural network is conducted by presenting a series of example patterns of associated input and output values. Initially, when a network is created, the connection weights and biases are set to random values. The performance of an ANN model is measured in terms of desired output and an error criterion. The output obtained at the end of each feedforward computation is compared with the target output and used to calculate a mean square error. An algorithm called backpropagation is then used to adjust the weights and biases until the mean square error is minimized. The network is trained by repeating this process several times. Once the ANN is trained, the prediction mode simply consists of propagating the data through the network (FIG. 3C), giving immediate results (FIG. 4). In the testing phase (prediction mode) the weights and biases may be held constant.

Various other pattern recognition techniques such as Principal Component Analysis (PCA) may also be used. PCA is essentially a data reduction technique by which a smaller number of variables are formed from a combination of original variables. For example, data reduction allows responses from seven sensors to be processed and displayed in two dimensions (FIG. 7). It is often easier for a user to interpret data that is displayed in fewer dimensions. FIG. 7 illustrates the vapor analysis of Benzene, Toluene, Ethylenzene, and p-Xylene (B, T, E, X) as sensed and processed using an embodiment. Three replicate analyses were run for each sample, and the sensor data was processed using PCA. The observed PCA plot shows four separate clusters representing the four samples (B, T, E and X).

Embodiments thus far described have been directed to detecting odors associated with soil borne contaminants; however, the ANN based processor can be used for measuring substantially any type of odor. For example, an alternative preferred embodiment can be used for evaluating the quality and type of coffee bean. An ANN based coffee-olfactometer may provide significant cost savings when used in place of conventional coffee quality assessment techniques such as using human based cupping or tasting. In addition, an ANN based olfactometer can produce results much faster than a human tester.

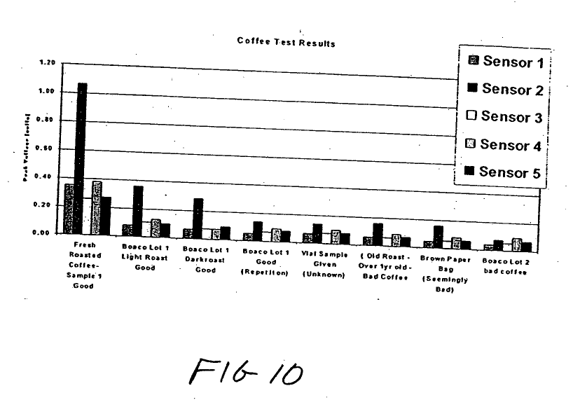

FIG. 8 illustrates how the ANN based coffee olfactometer is used to mimic the olfactory perceptions of a human coffee tester. FIG. 9 illustrates a schematic representation of a coffee-olfactometry system 80. System 80 contains a sealed coffee chamber 82 containing a perforated bean rack 86 and a heating element 84. Heating element 84 may be used for increasing the aroma associated with a plurality of coffee beans 88. The vapor created by beans 88 propagates through piping 90 to a second sealed chamber referred to as the sensor chamber 92. While the embodiment of FIG. 9 is shown with two chambers 82, 92, alternative embodiments can employ a single chamber. Sensor chamber 92 includes a plurality of sensors 94 for measuring the vapor received from coffee chamber 82. Sensors 94 may be selected such that a sensor output changes in frequency when a target vapor is detected such as would occur with a surface acoustic wave sensor or the sensors may be selected such that a change in conductivity is realized when a target vapor is detected such as would occur when using a chemo-resistive sensor. Each sensor 94 in a sensor array may be coated using a different substance to further provide unique response characteristics thereto for differing odors, or vapors. Sensor chamber 92 may further include a temperature sensor, a humidity sensor, a flow meter, and a pressure sensor for monitoring the environment inside the apparatus. Sensor chamber 92 can also include an exhaust fan 101 for facilitating movement of odors across sensor array 94.

Data cable 96 couples the output of each sensor with the ANN-based processor 98. Processor 98 processes sensor data using an ANN as previously described in conjunction with the soil sampling embodiment. The output of processor 98 can be made available to a display 100 for presentation to a user.

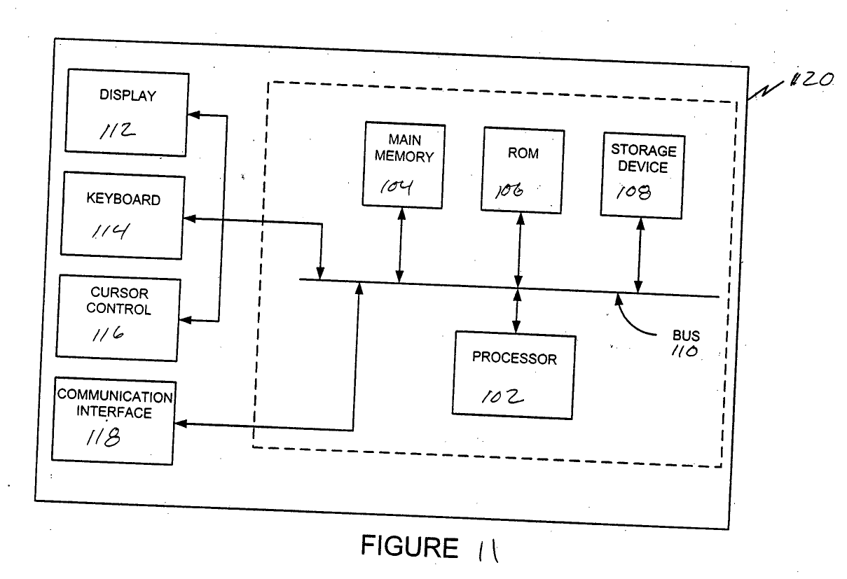

The coffee olfactometer is trained and verified by comparing results obtained from system 80 with results from one or more trained human testers. In addition, gas chromatography and gas chromatography/mass spectrometry can be used to identify specific compounds that contribute to particular flavors and aromas. Based on training as indicated above, the results shown in FIG. 10 were obtained.

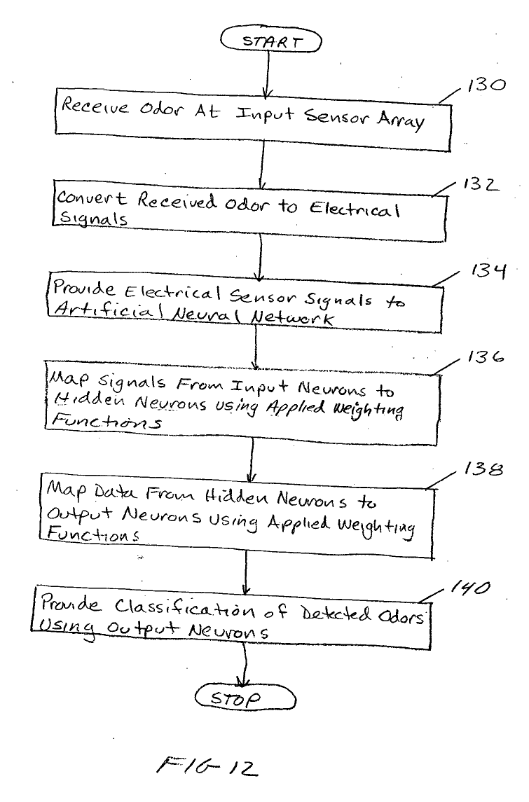

The ANN based soil olfactometer and coffee olfactometer can be implemented using a general purpose computing architecture such as that illustrated in FIG. 11. FIG. 11 illustrates the computer 120 in more detail. The exemplary computer 120 includes a processor 102, main memory 104, read only memory (ROM) 106, storage device 108, bus 110, display 112, keyboard 114, cursor control 116, and communication interface 118.

Processor 102 may be any type of conventional processing device that interprets and executes instructions. Main memory 104 may be a random access memory (RAM) or a similar dynamic storage device. Main memory 104 stores information and instructions to be executed by processor 102. Main memory 104 may also be used for storing temporary variables or other intermediate information during execution of instructions by processor 102. ROM 106 stores static information and instructions for processor 102. It will be appreciated that ROM 106 may be replaced with some other type of static storage device. Data storage device 108 may include any type of magnetic or optical media and its corresponding interfaces and operational hardware. Data storage device 108 stores information and instructions for use by processor 102. Bus 110 includes a set of hardware lines (conductors, optical fibers, or the like) that allow for data transfer among the components of computer 120.

Display device 112 may be a liquid crystal display cathode ray tube (CRT), or the like, for displaying information to a user. Keyboard 114 and cursor control 116 allow the user to interact with computer 120. Cursor control 116 may be, for example, a mouse. In an alternative configuration, keyboard 114 and cursor control 116 can be replaced with a microphone and voice recognition means to enable the user to interact with computer 120.

Communication interface 118 enables computer 120 to communicate with other devices/systems via any communications medium. For example, communication interface 118 may be a modem, an Ethernet interface to a LAN, or a printer interface. Alternatively, communication interface 118 can be any other interface that enables communication between the computer 120 and other devices or systems.

Computer 120 performs operations necessary to complete desired actions in response to processor 102 executing sequences of instructions contained in, for example, memory 104. Such instructions may be read into memory 104 from another computer-readable medium, such as a data storage device 108, or from another device via communication interface 118. Execution of the sequences of instructions contained in memory 104 causes processor 102 to perform a method for receiving and identifying odors using an artificial neural network. For example, processor 102 may execute instructions to perform the functions of mapping data from a plurality of input neurons to a plurality of hidden neurons and then to a plurality of output neurons. Alternatively, hard-wired circuitry may be used in place of or in combination with software instructions to implement the present invention. Thus, the present invention is not limited to any specific combination of hardware circuitry and software.

FIG. 12 illustrates an exemplary method for using an artificial neural network based olfactometer. The method begins when an odor is received at the sensor array 60 or 94 (per step 130). The received odor is converted to a plurality of electrical signals by the respective sensor array (per step 132). These electrical signals are provided to an artificial neural network where they serve as input to the input neurons, respectively (per step 134). The sensor signals are mapped from the input neurons to a plurality of hidden neurons using a plurality of applied weighting functions (per step 136). The neural network is configured such that each connection between the input neurons and the hidden neurons has its own selectable weighting function. The hidden neurons perform processing on the received data before making the data available to a plurality of output neurons (per step 138). The data links from the hidden neurons each have a separate unique weighting function. The output neurons may each provide a result to a user that is indicative of classification of a detected odor (per step 140).

As shown by the illustrated embodiments herein, the artificial neural network based olfactometer is capable of being trained to detect substantially any identifiable odor. Embodiments of the invention are therefore applicable to essentially any industry or application where automated detection and classification of odors is desired.

The claims should not be read as limited to the described order or elements unless stated to that effect. Therefore, all embodiments that come within the scope and spirit of the following claims and equivalents thereto are claimed as the invention.

What is claimed:

1. An apparatus for detecting a subsurface substance comprising:
a probe housing;
a sensor array mounted on said housing, the array including a plurality of sensors that receive a fluid mixture, said sensor array producing a plurality of sensor output signals in response to said received fluid mixture; and
a pattern recognition system having an olfactory pattern response that receives said plurality of sensor output signals and that produces a set of outputs.

2. The apparatus of claim 1 wherein said probe housing is a module to be advanced through a soil column.

3. The apparatus of claim 2 wherein said module is deployed using a push rod.

4. The apparatus of claim 1 wherein said fluid mixture contains an odorant.

5. The apparatus of claim 1 wherein said sensor and neural network are connected to detect a volatile organic compound.

6. The apparatus of claim 5 wherein said volatile organic compound is a hydrocarbon selected from the group consisting of polyaromatic hydrocarbon (PAH) and chlorinated hydrocarbons.

7. The apparatus of claim 1 wherein said neural network is an artificial neural network.

8. The apparatus of claim 7 wherein said olfactory pattern response comprises a plurality of learned reference data that is compared with the sensor output.

9. An apparatus for detecting an odor comprising:
a sample chamber for retaining a sample from which an odor is obtained;
a sensor array communicatively coupled to said sample chamber for receiving said odor and for generating a plurality of output signals in response to said odor; and
a pattern recognition system that processes said output signals to classify said odor.

10. The apparatus of claim 10 wherein said sample comprises a coffee bean or product thereof.

11. The apparatus of claim 9 wherein the pattern recognition system is a neural network.

12. The apparatus of claim 11 wherein said neural network is an artificial neural network.

13. The apparatus of claim 12 wherein data received from a human coffee taster is used to facilitate training said artificial neural network.

14. The apparatus of claim 13 wherein said artificial neural network generates a result indicative of a coffee type.

15. The apparatus of claim 13 wherein said artificial neural network generates a result indicative of coffee quality.

16. A method for classifying an odor comprising:
receiving said odor at a subsurface sensor array, said sensor array producing an output signal in response to receiving said vapor; and
processing said output signal using a pattern recognition system, said pattern recognition system generating a result indicative of an odorant with which said odor is associated.

17. The method of claim 16 further comprising sensing a volatile organic compound.

18. The method of claim 17 further comprising sensing said volatile organic compound located in a soil column.

19. The method of claim 18 further comprising providing said sensor array is in a probe deployed said soil column to exposed said sensor array to the volatile organic compound.

20. The method of claim 16 further comprising providing said pattern recognition system including a neural network.

21. The method of claim 20 further comprising providing said neural network including an artificial neural network.

22. The method of claim 21 wherein said odorant is a soil contaminant.

23. The method of claim 19 further comprising providing a probe including a metal cone.

24. The method of claim 16 further comprising measuring temperature data, pressure data or conductivity data.
